SEQUENCE LISTING (28 sequences)

SEQ ID NO: 1 - protein, 4 aa, Artificial
  Description of Artificial Sequence Synthetic
XGXL

SEQ ID NO: 2 - protein, 4 aa, Artificial
  Description of Artificial Sequence Synthetic
XGXG

SEQ ID NO: 3 - protein, 5 aa, Artificial
  Description of Artificial Sequence Synthetic
XGXGL

SEQ ID NO: 4 - protein, 5 aa, Artificial
  Description of Artificial Sequence Synthetic
XGXWG

SEQ ID NO: 5 - protein, 5 aa, Artificial
  Description of Artificial Sequence Synthetic
XGXXG

SEQ ID NO: 6 - protein, 5 aa, Artificial
  Description of Artificial Sequence Synthetic
XGXGG

SEQ ID NO: 7 - protein, 5 aa, Artificial
  Description of Artificial Sequence Synthetic
XGXLG

SEQ ID NO: 8 - protein, 4 aa, Artificial
  Description of Artificial Sequence Synthetic
XGXL

SEQ ID NO: 9 - protein, 4 aa, Artificial
  Description of Artificial Sequence Synthetic
XGXW

SEQ ID NO: 10 - protein, 4 aa, Artificial
  Description of Artificial Sequence Synthetic
XGXX

SEQ ID NO: 11 - protein, 4 aa, Artificial
  Description of Artificial Sequence Synthetic
XGXG

SEQ ID NO: 12 - protein, 5 aa, Artificial
  Description of Artificial Sequence Synthetic
XGXWG

SEQ ID NO: 13 - protein, 5 aa, Artificial
  Description of Artificial Sequence Synthetic
XGXXG

SEQ ID NO: 14 - protein, 5 aa, Artificial
  Description of Artificial Sequence Synthetic
XGXGG

SEQ ID NO: 15 - protein, 5 aa, Artificial
  Description of Artificial Sequence Synthetic
XGXLG

SEQ ID NO: 16 - protein, 4 aa, Artificial
  Description of Artificial Sequence Synthetic
XGLW

SEQ ID NO: 17 - protein, 4 aa, Artificial
  Description of Artificial Sequence Synthetic
XGXW

SEQ ID NO: 18 - protein, 4 aa, Artificial
  Description of Artificial Sequence Synthetic
XGMW

SEQ ID NO: 19 - protein, 5 aa, Artificial
  Description of Artificial Sequence Synthetic
XGXWG

SEQ ID NO: 20 - protein, 5 aa, Artificial
  Description of Artificial Sequence Synthetic
XGMWG

SEQ ID NO: 21 - protein, 4 aa, Artificial
  Description of Artificial Sequence Synthetic
LGXL

SEQ ID NO: 22 - protein, 4 aa, Artificial
  Description of Artificial Sequence Synthetic
LGXG

SEQ ID NO: 23 - protein, 5 aa, Artificial
  Description of Artificial Sequence Synthetic
LGXLG

SEQ ID NO: 24 - protein, 5 aa, Artificial
  Description of Artificial Sequence Synthetic
LGXGG

SEQ ID NO: 25 - protein, 4 aa, Artificial
  Description of Artificial Sequence Synthetic
LGLL

SEQ ID NO: 26 - protein, 4 aa, Artificial
  Description of Artificial Sequence Synthetic
LGLW

SEQ ID NO: 27 - protein, 5 aa, Artificial
  Description of Artificial Sequence Synthetic
LGLLG

SEQ ID NO: 28 - protein, 5 aa, Artificial
  Description of Artificial Sequence Synthetic
LGLWG

(19) United States
(12) Patent Application Publication
(10) Pub. No.: US 2003/0195152 A1
(43) Pub. Date: Oct. 16, 2003

(54) Polymeric conjugates of antitumor agents

(76) Inventors: Antonio Suarato, Milan (IT); Francesco Angelucci, Milan (IT); Michele Caruso, Milan (IT); Alessandro Scolaro, Milan (IT); Daniele Volpi, Cornaredo (IT); Moreno Zamai, Milan (IT)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/333,619

(22) PCT Filed: Jul. 9, 2001
(86) PCT No.: PCT/EP01/07883

(30) Foreign Application Priority Data
Jul. 25, 2000 (GB) 00182402

(51) Int. Cl. A61K 38/00; C07K 14/00

(52) U.S. Cl. 514/12; 530/350

(57) ABSTRACT

Water soluble polymeric conjugates of antitumor agents of formula (A) P-[W₂]ₚ-S₀-[W₁]ᵣ-[D] wherein: P is a water soluble polymer; [W₁] is a residue of formula —HN-Z₁-CO— in which Z₁ represents a linear or branched C2-C12 alkylene chain or the residue of formula —C6HC—CH2—O—; [W₂] is a residue of formula —HN-Z2-CO— in which Z2 represents a C2-C12 linear or branched alkylene chain; p and r are 0 or 1; S0 is a peptide that selectively is cleaved at the tumor site mainly by the action of the matrix metalloproteinases gelatinase; [D] is the residue of an antitumor agent. The conjugates possess enhanced antitumor activity and decreased toxicity with respect to the free drug. A process for their preparation, useful intermediates and pharmaceutical compositions containing them are also described.

The present invention relates to polymeric conjugates of antitumor agents releasing the free active drug at tumor site mainly by the action of matrix metalloproteinases (MMPs). The drugs include antitumor agents such as cytotoxics or inhibitors of enzymes involved in the tumor growth and spread. A first object of the present invention is to provide a polymeric drug-conjugate that selectively releases the active drug at the tumor site mainly by the action of the matrix metalloproteinases gelatinase: A correlation between tumor secretion of matrix metalloproteinases, particularly MMP2 or gelatinase A, and experimental metastasis has been reported (Liotta, L. A. et al., J. Natl. Cancer Inst., 81: 556, 1989; Nakajima, M. et al., Cancer Res., 47: 4869, 1987). MMP2 was found to be highly expressed in stromal cells surrounding the invading front of metastasizing tumors (Salo, T. et al., J. Biol. Chem., 258: 3058, 1983; Reponen, P. et al., ibid. 267: 7856, 1992). This enzyne is expressed in a variety of tumor types including skin (Pyke, C. et al., Cancer Res., 52: 1336, 1992), lung (Kodate, M. et al., Pathol. International, 47: 461, 1997 Nawrocki, B. et al., International J. of Cancer, 72: 556, 1997), gastric (Endo, K. et al., Anticancer Research, 17: 225,3 1997), colorectal (Liabakk, N. et al., Cancer Res., 56: 190, 1996; Pyke, C. et al., Am. J. Pathol., 142: 359, 1993), breast (Gilles, C. et al., Laboratory Invest. 76: 651, 1997; Davies, B. et al., Br. J. Cancer, 67: 1126, 1993), prostate (Stearns, M. E. et al., Oncology Res., 8: 69, 1996), ovarian (Fishman, D. et al., Invasion & Metastasis, 16: 150, 1996) and bladder (Miyake H. et al., J. of Urology, 157: 2351, 1997).

Different classes of polymeric conjugates of antitumor agents have been already disclosed in PCT Publication No. WO99/17805 and WO99/17804 and in the patents U.S. Pat. No. 5,773,522 and U.S. Pat. No. 5,618,790.

The present invention provides a polymeric drug-conjugate of general formula (A)

P-[W₂]ₚ-S₀-[W₁]_{r-[D]} (A)

wherein:

P is a water soluble polymer;

[W₁] is a residue of formula —HN-Z₁-CO— in which Z, represents a linear or branched C₂-C₁₂ alkylene chain or the residue of formula —C₆H₄—CH₂—O—;

[W₂] is a residue of formula —HN-Z₂-CO— in which Z₂ represents a C₂-C₁₂ linear or branched alkylene chain;

p and r are 0 or 1;

S₀ is a peptide residue selectively cleavable at the tumor site by the action of matrix metalloproteinases and

[D] is the residue of an antitumor agent.

Thus the present invention provides polymer-conjugates of antitumor agents of general formula (A) that may be cleaved by matrix metalloproteinase, particularly gelatinase, to release intermediates of formula S₁-[W₁]ᵣ-[D], wherein S₁ is a peptide derived from S₀ and [W₁], [D] and r are above defined, from which the antitumor agent D is released spontaneously or by the action of proteolytic enzymes present in the tumor tissue.

Another aspect of the present invention is to provide a method of treating solid tumors, which comprises administration of the novel polymeric drug-conjugates of general formula (A).

Preferably P is a water soluble polymer such as poly-glutammic acid, carboxylated dextranes, carboxylated polyethylenglycols or a polymer based on hydroxypropyl-methacrylamide. Most preferably P is a polymer based on N-(2-hydroxypropyl) methacryloylamide (EPMA).

Preferably [W₁] is absent, i.e. r=0, or is the self-imolative p-aminobenzyloxycarbonyl linker [see: J. Med. Chem vol 24, 479 (1991); PABC, -pHN—C₆H₄—CH₂-O—CO—]. A C₂-C₁₂ alkylene chain which Z₁ and Z₂ may represent comprises a residue of the formula (CH₂)₃—; —CH₂—C(CH₃)₂—CH₂; and —(CH₂)₅—. Preferably [W₂] is the residue of 6-aminohexanoic acid, Z₂=—(CH₂)₅—.

The peptide S₀ comprises sequences from four to five natural or synthetic amino acids. Preferably the peptide S₀ represents Met(O)-Gly-Cys(Bn)-Leu (SEQ ID NO: 1), Met(O)-Gly-Cys(Bn)-Gly (SEQ ID NO: 2), Met(O)-Gly-Cys(Bn)-Gly-Leu (SEQ ID NO: 3), Met(O)-Gly-Cys(Bn)-Trp-Gly (SEQ ID NO: 4), Met(O)-Gly-Cys(Bn)-pFF-Gly (SEQ ID NO: 5), Met(O)-Gly-Cys(Bn)-Gly-Gly (SEQ ID NO: 6), Met(O)-Gly-Cys(Bn)-Leu-Gly (SEQ ID NO: 7), Smc-Gly-Cys(Bn)-Leu (SEQ ID NO: 8), Smc-Gly-Cys(Bn)-Trp (SEQ ID NO: 9), Smc-Gly-Cys(Bn)-pFF (SEQ ID NO: 10), Smc-Gly-Cys(Bn)-Gly (SEQ ID NO: 11), Smc-Gly-Cys(Bn)-Trp-Gly (SEQ ID NO: 12), Smc-Gly-Cys(Bn)-pFF-Gly (SEQ ID NO: 13), Smc-Gly-Cys(Bn)-Gly-Gly (SEQ ID NO: 14), Smc-Gly-Cys(Bn)-Leu-Gly (SEQ ID NO: 15), Smc-Gly-Leu-Trp (SEQ ID NO: 16), Smc-Gly-Tha-Trp (SEQ ID NO: 17), Smc-Gly-Met-Trp (SEQ ID NO: 18), Smc-Gly-Tha-Trp-Gly (SEQ ID NO: 19), Smc-Gly-Met-Trp-Gly (SEQ ID NO: 20), Leu-Gly-Cys(Bn)-Leu (SEQ ID NO: 21), Leu-Gly-Cys(Bn)-Gly (SEQ ID NO: 22), Leu-Gly-Cys(Bn)-Leu-Gly (SEQ ID NO: 23), Leu-Gly-Cys(Bn)-Gly-Gly (SEQ ID NO: 24), Leu-Gly-Leu-Leu (SEQ ID NO: 25), Leu-Gly-Leu-Trp (SEQ ID NO: 26), Leu-Gly-Leu-Leu-Gly (SEQ ID NO: 27), Leu-Gly-Leu-Trp-Gly (SEQ ID NO: 28).

The most preferred peptide sequences S₀ are:

Met(O)-Gly-Cys(Bn)-Leu (SEQ ID NO: 1), Met(O)-Gly-Cys(Bn)-Gly (SEQ ID NO: 2), Met(O)-Gly-Cys(Bn)-Gly-Gly (SEQ ID NO: 6), Met(O)-Gly-Cys(Bn)-Leu-Gly (SEQ OD NO: 7), Smc-Gly-Cys(Bn)-Leu (SEQ ID NO: 8), Smc-Gly-Cys(Bn)-Gly (SEQ ID NO: 11), Smc-Gly-Cys(Bn)-Gly-Gly (SEQ ID NO: 14), Smc-Gly-Cys(Bn)-Leu-Gly (SEQ ID NO: 15), Leu-Gly-Cys(Bn)-Leu (SEQ ID NO: 21), Leu-Gly-Cys(Bn)-Gly (SEQ ID NO: 22), Leu-Gly-Cys(Bn)-Leu-Gly (SEQ ID NO: 23), Leu-Gly-Cys(Bn)-Gly-Gly (SEQ ID NO: 24), Leu-Gly-Leu-Leu (SEQ ID NO: 25), Leu-Gly-Leu-Leu-Gly (SEQ ID NO: 27). In the present specification, Leu is leucine, Trp is tryptophan, Met is methionine, Met(O) is methionine sulfoxide, Cys(Bn) is S-benzyl-cysteine, Smc is S-methylcysteine, Tha is thienyl alanine, pFF is p-fluorophenylglycine, Gly is glycine.

The drug residue [D] is the residue of an antitumor agents bearing functional groups for the attachment of the linker W₁ or the peptide S₀. Such functional groups comprise hydroxy, carbonyl, carboxy, primary or secondary amino groups and sulfidryl. Preferred antitumor agents include cytotoxic agents belonging to the class of vinca alkaloids, anthracyclines, taxanes, cytotoxic nucleosides, camptothecins, podophyllotoxins, alkylating agents. Members of those classes include, for example, 4deacetyl-vinblastine, 4-deacetyl-vincristine, vindesine, doxorubicin, 4′-epidoxorubicin, daunorubicin, 4demethoxy-daunorubicin, 4′-deoxy-4′-iododoxorubicin, 3′-(2-methoxymorpholino) doxorubicin, paclitaxel, docetaxel, 5-fluorouracil, camptothecin, 7-ethyl-10-hydroxycamptothecin, 9-aminocamptothecin, etoposide, estramustine. Other antitumor drugs of the present invention include inlibitors of enzymes involved in the tumor growth and spread.

The present invention also provides methods for preparing drug-conjugates of general formula (A) which comprise:

reacting a compound of general formula (1) or the corresponding salt derivatives of formula (1′):

H-[W₂]ₚ-S₀-[W₁]ᵣ-[D] (1)

RH H-[W₂]ₚ-S₀-[W₁]ᵣ-[D] (1′)

wherein [W₁], [W₂], p, r, S₀ and [D] are as above defined; and RH is an acid, with a polymer P1 bearing suitable functional groups for the coupling with compounds (1) or (1′). Suitable functional groups on polymer P1 for the attachment to compounds (1) or (1′) comprise carboxyl groups or activated carboxyl groups such as p-nitrophenyl ester or imidazoyl ester. Preferably RH in (1′) represents HCl or CF₃COOH.

Compounds of formula (1) and the corresponding salt derivatives (1′) are also provided by the present invention. They can be prepared following different synthetic methods. One method comprises removing the N-protecting group, preferably in acidic conditions, from a derivative of formula (2);

R₂-[W₂]ₚ-S₀-[W₁]ᵣ-[D] (2)

wherein [W₁], [W₂], p, r, [D] and S₀ are as above defined, and R₂ represents an amino-protecting group, such as Boc (tert-butoxycarbonyl), Fmoc, triphenylsilyl, diphenylmethylene or triphenylmethyl, to give a compound of general formula (1) or (1′) as defined above, optionally converting a compound of general formula (1′) into the corresponding free amino derivative (1) by mild basic treatment.

Derivatives of formula (2) are preferably prepared with a method which comprises reacting a compound of formula (3) with a compound of the formula (4)

H-Sₓ-[W₁]ᵣ-[D] (3)

R₂-[W₂]ₚ-S_{y}-R₁ (4)

wherein [W₁], [W₂], R₂, p, r, are as above defined, R₁ is hydroxy group or the residue of an activated ester such as p-nitrophenoxy or N-hydroxysuccinimido group or a halogen such as chlorine and Sₓ, S_{y} are independently an amino acid or peptide residue characterized that, when linked together, form a peptide residue S₀ as above defined, optionally in presence of a condensing agent, to give a derivative of formula (2) as defined above. Preferably S_{y} represents Met(O)-Gly, Smc-Gly or Leu-Gly, and Sₓ represents Cys(Bn)-Leu, Cys(Bn)-Gly, Cys(Bn)-Gly-Leu, Cys(Bn)-Trp-Gly, -Cys(Bn)-pFF-Gly, Cys(Bn)-Gly-Gly, Cys(Bn)-Leu-Gly, Cys(Bn)-Trp, Cys(Bn)-pFF, Leu-Trp, Tha-Trp, Met-Trp, Tha-Trp-Gly, Met-Trp-Gly, Leu-Leu, Leu-Leu-Gly or Leu-Trp-Gly.

Derivatives of formula (2) may be also prepared with a method which comprises reacting an anticancer agent D bearing a functional group for the attachment to the linker W₁ or the peptide S₀, with N-protected derivatives of formula (5)

R₂-[W₂]ₚ-S₀-[W₁]ᵣ-R₁ (5)

wherein [W₁], [W₂], R₂, R₁, p, r, and S₀ are as above defined, to give a derivative of formula (2) as defined above.

Alternative methods for the preparation of compounds of formula (2) as defined above comprise for example:

a) reacting the drug D with a compound of formula (6)

R₂-[W₁]ᵣ-R₁ (6)

wherein W₁, r, R₁ and R₂ are as previously defined,

b) removing the protecting group from the resultant compound of formula (7)

R₂-[W₁]ᵣ-[D] (7)

wherein W₁, r, R₂ and [D] are as previously defined,

c) reacting the resultant derivatives of formula (8):

H-[W₁]ᵣ-[D] (8)

wherein W₁, r and [D] are as previously defined, with N-protected derivatives of formula (9),

R₂-[W₂]ₚ-S₀-R₁ (9)

wherein W₂, R₁, R₂, p and S₀ are as previously defined,

to give a derivative of formula (2) as defined above. Another method for the preparation of compounds of formula (2) comprises reacting the free base (10)

H-S₀-[W₁]ᵣ-[D] (10)

wherein W₁, [D], r and S₀ are as previously defined, with N-protected and activated compound of formula (11)

R₂-[W₂]ₚ-R_{1 (}11)

wherein W₂, p, R₁ and R₂ are as previously defined, to give a compound of formula (2) as defined above.

The preparation of the compounds of formula (4), (5) and (9) follows procedures known for the preparation of peptides. For example by using solid phase synthesis through a stepwise addition of N-protected amino acids to a growing chain attached to a solid resin, such as Wang resin. Preferably the N-protecting group is Fmoc. Thus the C-terminus of N-protected amino acid is linked to the resin in aprotic organic solvents such as methylene chloride in presence of organic base such as diisopropylethylamine (DIPEA). The completion of chain elongation is accomplished by the standard repetition of the deprotection/coupling cycle. Preferably the Fmoc protecting groups are deblocked with piperidine 20% in N-methyl-2-pyrrolidone and coupling steps are performed with TBTU, HOBt, DIPEA in N-methyl-2-pyrrolidone. Resin cleavage may be accomplished with a mixture of methylene chloride, acetic acid, trifluoroacetic acid (3/1/1 v/v) or methylene chloride, trifluoroacetic (99/1 v/v).

The compounds of formula (3) and (10) may be prepared by reacting compounds (8) as defined above sequentially with N-protected amino acids or peptides. The preparation of starting compounds of formula (6), (7), (8) and (11) follows known procedures. The preparation of compounds of formula (1) follows synthetic procedures similar to those described in our previous PCT Publication No. WO99/17805 and WO99/17804 and in the patents U.S. Pat. No. 5,773,522 and U.S. Pat. No. 5,618,790.

The preparation of drug-conjugates of formula (1) and (1′), intermediates for the preparation of polymer-drug-conjugates (A), is illustrated in the following synthetic Scheme. For example in Scheme 1 is illustrated the preparation of 7-ethyl-10-hydroxy-20-O-[6-aminohexanoyl-(methionyl-sulfoxide)-glycyl-(S-benzyl-cysteinyl)-leucyl-glycyl]-camptothecin salt derivative (1′a). The synthetic process comprises sequential attachments of N-protected amino acids to 7-ethyl-10-hydroxy-camptothecin (D₁: 12). In particular (12) is reacted with a molar excess, for example up to three mol. equivalent, of N-t-butoxycarbonyl-glycine p-nitrophenyl ester (Boc-Gly-ONP) in anhydrous non-protic solvent such as dimethylsulfoxide, in presence of an activating agent such as 4-dimethylamino pyridine (DMAP), under argon. In this manner, the protected amino acid is introduced at both hydroxylated positions C-10 and C-20 of compound (12). The reaction can typically be effected for from 8 to 48 hours. The reaction is typically carried out at a temperature from 15 to 40° C. The substituent group at position C-10 is removed in presence of a secondary amine such as morpholine, 1-amino-prolinol, to give the mono-substituted N-Boc-glycyl-derivative at C-20 (13). The amino protecting group may be removed by acidic treatment, such as 1.5 N HCl in acetic acid or 95% aqueous trifluoroacetic acid for from 10′ to 6 hours at a temperature of from 10° to 30° C.; preferably for half an hour at room temperature to give the 20-O-glycyl-derivative (14′) in the acid-salt form. The second amino acid leucine may be introduced by reacting compound (14′) with molar excess, for example up to two mol. equivalent of N-t-butoxycarbonyl-leucine (Boc-Leu-OH) in anhydrous non-protic solvent, preferably dimethylformamide, in presence of condensing agents such as 1-hydroxybenzotriazole (HOBt), O-(benzotriazol-1-yl)-N,N,N′,N′-tetramethyluronium tetra-fluoborate (TBTU) and diisopropylamine (DIPEA). The reaction can typically be effected for from 8 to 48 hours. The reaction is typically carried out at a temperature from 15 to 40° C. Treatment with morpholine, followed by acidic displacement of the N-protecting group of compound (15) affords 7-ethyl-10-hydroxy-20-O-(leucyl-glycyl)-camptothecin (16′). This compound is treated in sequence with N-t-butoxycarbonyl-S-benzylcysteine to afford compound (17) and N-t-butoxycarbonyl-6-aminohexanoyl-(methionyl-sulfoxide)-glycyl in the same conditions previously described for the attachment of leucine to produce the final intermediate (1′a) in the form of salt, such as trifluoroacetate salt derivative.

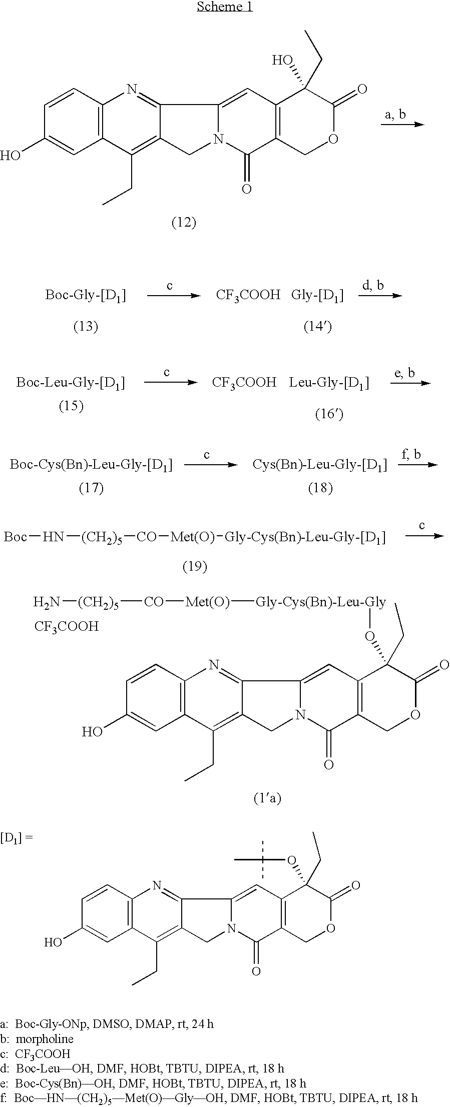

Scheme 2 illustrates the process for the preparation conjugate of 4-deacetyl vinblastine bearing the sequence 6-aminohexanoyl-leucyl-glycyl-leucyl-leucyl (1c). The process starts from vinblastine sulphate (20) which is firstly converted into free base by alkaline treatment and then deacylated to compound (21) at position C-4 by using sodium methylate in dry methanol. Reaction of 4-deacetyl-vinblastine (21) with N-protected derivafive of leucyl, such as Fmoc derivative, in the form of acyl chloride affords Fmoc-leucyl-derivative (22). Removal of the N-protecting group to form compound (23) is tipically carried out in a mixture of piperidine in dimethylformamide. Attachment of 6-aminohexanoyl-leucyl-leucyl-glycyl is tipically performed as previously described to produce (1c′) after removal of the N-protecting group and salt formation.

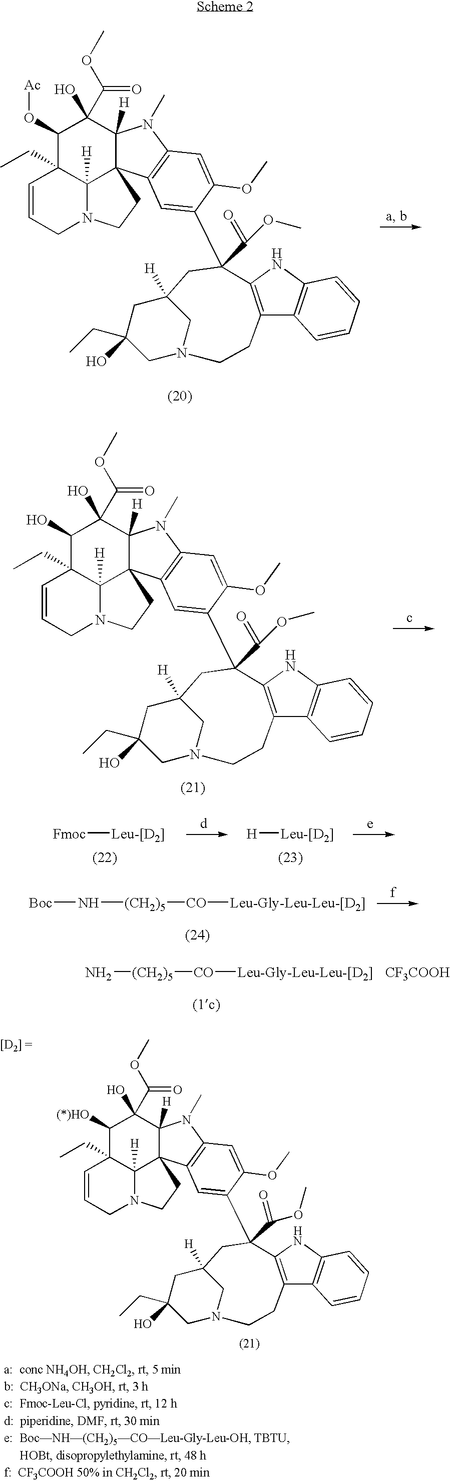

As previously illustrated compounds of formula (1) or (1′) are useful intermediates for the preparation of polymeric anticancer agents of formula (A) which are the objective of the present invention.

Without limiting the scope of the present invention hereinafter are reported examples of polymeric drug-conjugates of formula (A) in which the water soluble polymer P is based on N-(2-hydroxypropyl)methacryloylamide (HPMA).

In such case the polymeric drug-conjugates indicated as (A) comprise a soluble polymer P′ consisting of:

(i) from 85 to 97 mol % of N-(2-hydroxypropyl)methacryloylamide units represented by formula (25)

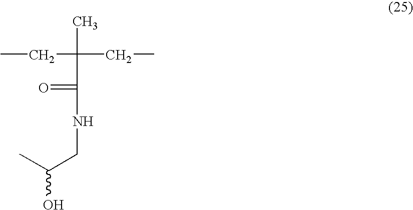

(ii) from 3 to 15 mol % of units represented by formula (26)

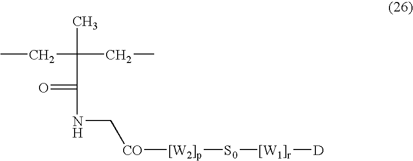

in which [W₁], [W₂], p, r, [D] and S₀ are as above defined,

(iii) from 0 to 12 mol % of N-methacryloyl-glycine or N-(2-hydroxypropyl) methacryloyl-glycinamide units represented by formula (27)

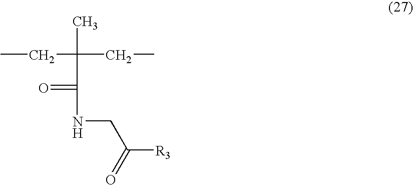

wherein R₃ represents a hydroxy group or a residue of formula —NH—CH₂—CH(OH)—CH₃.

This polymeric drug-conjugate (A) may also be represented as follows:

[(25)]ₓ; [(26)]_{y}; [(27)]_{z} wherein (25), (26) and (27) are units of the formula as above defined, and x is from 85 to 97 mol %, y is from 3 to 15 mol % and z is from 0 to 12 mol %.

Preferably, this polymeric drug-conjugate (A) as above defined contains the N-(2-hydroxypropyl) methacryloyl amide units represented by the formula (25) in a proportion of 90% or more; more preferably 90%. The conjugate may also contain from 3 to 10 mol % of methacryloyl-glycyl-derivative units represented by the formula (26), more preferably 10 mol % of such units. Preferably (A) does not contain residues of formula (27), i.e. z is 0.

The present invention also provides a process for preparing drug-conjugates of formula (A) which comprises reacting compounds of general formula (A) with an activated polymer P1′ consisting essentially of:

(i) from 85 to 97 mol % of N-(2-hydroxypropyl)methacryloylamide units represented by formula (25) as above defined, and

(ii) from 3 to 15 mol % of N-methacryloyl-glycyl units represented by formula (28)

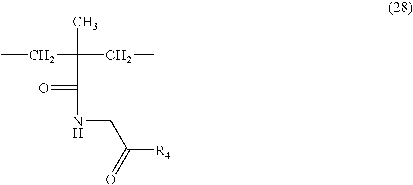

wherein R₄ is the residue of an active ester, and optionally displacing the remaining active ester groups with 1-amino-2-propanol.

Polymers of formula P1′ have been already described in Makromol. Chem. 178, 2159 (1977). Preferably, the reaction between a polymer (P1′) in which R₄ in formula (28) represents the residue of active ester and a compound of formula (1) to prepare the polymeric drug-conjugate (A) is carried out in an anhydrous polar organic solvent such as dimethylsulfoxide. The reaction can typically be carried out at temperature from 15 to 30° C., preferably at room temperature for 15 hours; then the aminolysis of the remaining active ester groups can be performed in the presence of 1-amino-2-propanol at room temperature, from 0.5 to 1 hour. The conjugate suitably is precipitated with ethyl acetate, dissolved in ethanol and precipitated with ethyl acetate.

For example polymer P1′ in which R₄ of formula (28) represents the residue of an active ester such as p-nitrophenol, provided at a concentration of 15% (w/v) in dry dimethylsulfoxide, is treated with compound of formula (1′a-1′c), 3% (w/v), in presence of a tertiary amine such as DPEA or triethylamine, at room temperature for 15 hours. Then 1-amino-2-propanol in DMF, 0.1% (w/v) is added and the reaction mixture is kept at room temperature for 8 hours. The resulting polymer drug-conjugates (A1a -A1c) can be precipitated with ethyl acetate, collected, washed with ethyl acetate, then dissolved with absolute ethanol at a concentration of 10% (w/v), treated with a sulfonic resin, filtered and precipitated again with ethyl acetate.

The process is illustrated in Scheme 3 and 4.

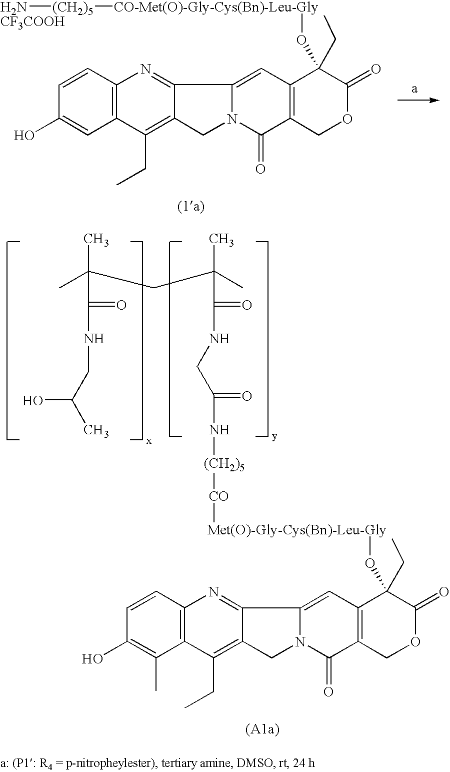

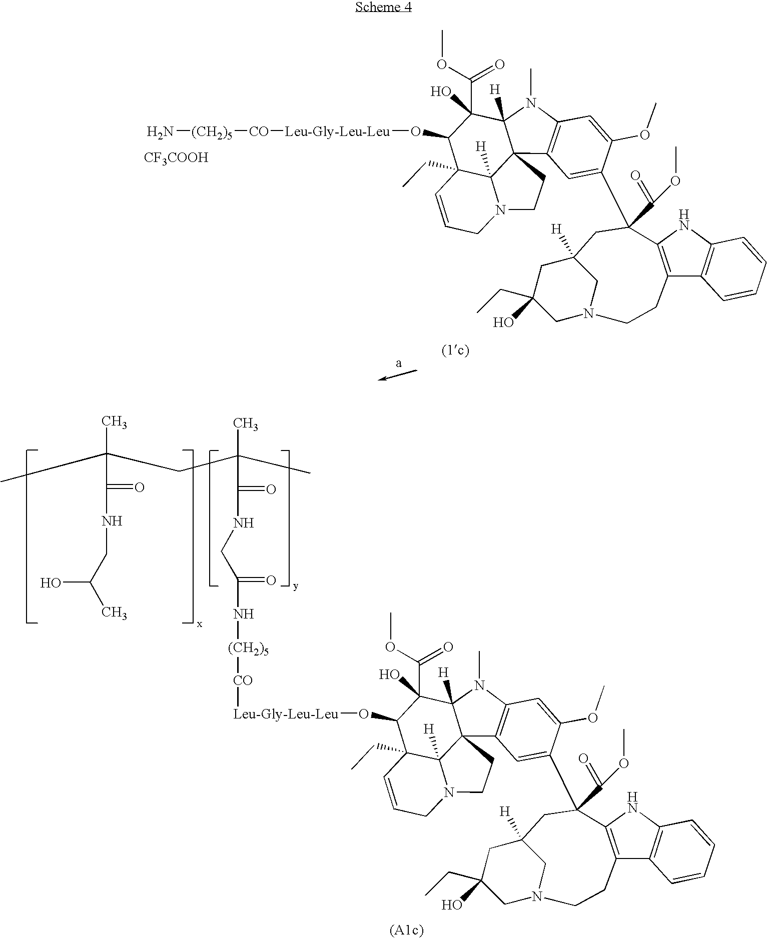

The content of active drug in polymeric conjugate of the invention is determined by HPLC or absorbency spectroscopy analysis.

The polymeric drug-conjugates of formula (A) are in the range of 5.000 to 45.000 molecular weight, preferably from 12.000 to 25.000. Compounds of formula (A) and other compounds of the invention are water-soluble and show enhanced antitumor activity and reduced toxicity in comparison with the free drug. They are useful in the treatment of leukemia and solid tumors, such as colon, colo-rectal, ovarian, mammary, prostate, lung, kidney and also melanoma tumors. A human can therefore be treated by a method comprising administering thereto a therapeutically effective amount of a polymeric conjugate of the invention. Thie condition of the human patient can thus be improved.

The dosage range adopted will depend on the route of administration and on the age, weight and condition of the patient being treated. The polymeric drug-conjugates of formula (A) as well as soluble salt derivatives of formula (1′) are typically administered by parenteral route, for example intramuscularly, intravenously or by bolus infusion. A suitable dose range is from 1 to 1000 mg of equivalent per m² body surface area of active drug, for instance from 10 to 500 mg/m².

The polymeric drug-conjugate (A) or soluble salt derivatives of formula (1′) may be formulated into a pharmaceutical composition together with a pharmaceutically carrier or diluent. Typically they are formulated for parenteral administration, for example by dissolution in water for injection or physiological saline.

### Enzyme Assay

The degradation of polymeric drug-conjugate of formula (A) in vitro was investigated in buffer and in the presence of several proteolytic enzymes (matrix metallo proteinases, MMPs, serine protease (elastase)) and in plasma.

Polymeric drug-conjugates (A) were dissolved in sterile distilled water at the standard concentration of 10 mM. Concentrations were calculated as equivalent of drug according to the polymer loading percentage (5-10 wt % drug). Compounds (A) were assayed in 50 mM Tris/HCl pH 7.4 buffer containing 0.15 M NaCl, 10 μM CaCl₂, 0.01 mM Zn acetate and 0.05% C₁₂ E₉. The polymeric drug-conjugates are equilibrated at 37° C. in buffer for 5 minutes at the concentrations varying from 5 to 1000 μM. Reactions are started by addition of enzymes (MMPs) to a final concentration of 50 μM. Enzymatic reactions aree stopped within 5% of hydrolysis of polymeric drug-conjugates by adding 0.05% TFA buffer (pH 2.5) and subsequently analyzed by RP-HPLC through a aquapore OD300 column.

The quantification of products of reaction is obtained by RP-HPLC. For example with a Perkin Elmer HPLC consisting of an ISS 200 autosampler, a Series 200LC pump, and a LC240 fluorescence detector, or, alternatively, a Waters HPLC consisting of 717-plus autosampler, a Model 600 pump and a Model 474 fluorimeter.

We found that the compounds of the formula (A) and (1′) of present invention selectively release the antitumor agent D in presence of gelatinase and are substantially stable in plasma and in presence of other proteolytic enzymes.

The following Examiples illustrate the invention.

### EXAMPLE 1

### 7-Ethyl-10-hydroxy-20-O-glycyl-camptothecin trifluoroacetate (14′)

7-Ethyl-10-hydroxy-camptothecin (12) (2.1 g, 5.3 mmol) was dissolved in anhydrous dimethylsulfoxide (50 ml) and added with N-t-butoxycarbonyl-glycine p-nitrophenyl ester (4.8 g, 16.2 mmol) and dimethylaminopyridine (0.8 g, 6.5 mmol). The reaction mixture was kept in argon atmosphere under stirring for 24 h at room temperature Afterwards morpholine (4.6 ml) was added and the reaction mixture was kept under stirring for 24 h at room temperature. Then methylene chloride (200 ml) and aqueous 0.5N HCl were added. The organic phase was separated and washed with water (3×200 ml). The organic solvent was removed under reduced pressure to give 7-ethyl-10-hydroxy-20-O-[t-butoxycarbonyl-glycyl]-camptothecin (13). TLC on kieselgel plate (Merck), eluting system methylene chloride/methanol (95/5 v/v) Rf=0.3.

Compound (13) was dissolved in trifluoroacetic acid (50 ml of 95% aqueous solution) and after one hour the solvent was removed under reduced pressure to give the title compound (14′, yield 3 g) in the form of trifluoroacetate salt derivative.

### EXAMPLE 2

### 7-Ethyl-10-hydroxy-20-O-(leucyl-glycyl)-camptothecin trifluoroacetate (16′)

A solution of dimethylformamide (50 ml) containing N-t-butoxycarbonyl-leucine (2.49 g, 10 mmol), 1-hydroxybenzotriazole (HOBt) (1.53 g, 10 mmol), O-(benzotriazol-1yl)-N,N,N′,N′-tetramethyluronium tetrafluoborate (TBTU) (3.21 g, 10 mmol) and diisopropylethylamine (DIPEA) (3.5 ml, 20 mmol) was mixed after 30 min. with a solution of compound (14′) (3 g, 5.28 mmol), prepared as described in Example 1, in dimethylformamide (20 ml) containing DIPEA (1.75 ml, 10 mmol). The reaction mixture was kept under stirring overnight at room temperature. Then morpholine (4.5 ml, 5.6 mmol) was added and the mixture was stirred for six hours. After that the solvent was evaporated under reduced pressure, the residue was dissolved with methylene chloride (200 ml) and washed with aqueous 0.5N HCl, aqueous 5% NaHCO₃ (200 ml) and water (2×200 ml). The organic phase was dried over anhydrous sodium sulphate, then the solvent was removed under reduced pressure to give 7-ethyl-10-hydroxy-20-O-[N-t-butoxycarbonyl-leucyl-glycyl]-camptothecin (16). TLC on kieselgel plate (Merck), eluting system methylene chloride/methanol (95/5 v/v) Rf=0.5. Compound (16) was dissolved in trifluoroacetic acid (50 ml of 95% aqueous solution) and after one hour the solvent was removed under reduced pressure to give the title compound ((16′), yield 3 g) which was collected with ethyl ether in the form of trifluoroacetate salt derivative.

### EXAMPLE 3

### 7-Ethyl-10-hydroxy-20-O-[-(S-benzyl-cysteinyl)-leucyl-glycyl]-camptothecin trifluoroacetate (18′)

Compound (16′: 3 g) was reacted with N-t-butoxycarbonyl-(S-benzyl-cysteine) following the same conditions described in Example 2 to give 7-ethyl-10-hydroxy-20-O-[N-t-butoxycarbonyl-(S-benzyl-cysteinyl)-leucyl-glycyl]-camptothecin (17) which was transformed into the title compound (18) as described in the Example 2.

### EXAMPLE 4

### 7-Ethyl-10-hydroxy-20-O-[6-aminohexanoyl-(methionyl-sulfoxide)-glycyl-(S-benzyl-cysteinyl)-leucyl-glycyl]-camptothecin trifluoroacetate (1′a)

The title compound was prepared following the same conditions described in Example 2 by mixing a solution of diniethylformamide (DMF, 5 ml) containing N-t-butoxycarbonyl-6-aminohexanoyl-(methionyl-sulfoxide)-glycine (0.358 g, 0.57 mmol), and a solution of compound (18) (0.257 g, 0.76 mmol) in dimethylformamide (2 ml), and then removing the Boc protecting group.

### EXAMPLE 5

### Copolymer of N-(2-hydroxypropyl)methacryloylamide, 7-ethyl-10-hydroxy-20-O-[N-methacryloyl-6-aminohexanoyl-(methionyl-sulfoxide)-glycyl-(S-benzyl-cysteinyl)-leucyl-glycyl]camptothecin and N-(2-hydroxypropyl)methacryloylglycinamide (A1a)

To a solution in anhydrous dimethylsulfoxide (4 ml) of polymer (P1′) (0.756 g), were added 7-ethyl-10-hydroxy-20-O-[6-aminohexanoyl-(methionyl-sulfoxide)-glycyl-(S-benzyl-cysteinyl)-leucyl-glycyl]-camptothecin trifluoroacetate (1′a) (0.23 g, 0.193 mmol), prepared as described in Example 4, and triethylamine (0.53 ml, 0.396 mmol). The reaction mixture was kept in argon atmosphere under stirring at room temperature for 24 h. Then 0.1 ml of a solution of 1-amino-2-propanol (0.3 ml) in dimethylformamide (10 ml) was added and stirring was continued for 8 h. After that ethyl acetate (200 ml) was added to the reaction mixture under stirring. The precipitate was collected, washed with ethyl acetate and dissolved with ethanol (8 ml) and treated with sulphonic resin (DOWEX 50-sulfonic acid) for 30 min. The solution was filtered and precipitated with ethyl acetate. The resultant solid was washed with ethyl ether and dried at constant weight to give the title compound (A1a). Yield 0.8 g; Mw 20.800, polydispersity 1.48, loading of 7-ethyl-10-hydroxy-camptothecin 7.5% (w/w %).

### EXAMPLE 6

### 4-deacetyl-4-O-Leucyl-vinblastine (23)

To a solution of vinblastine sulphate (20) (1.5 g, 1.65 mmol) in water (15 ml) was added methylene chloride (15 ml) and conc. ammonia (1.5 ml) under vigorous stirring. After 5 minutes the mixture was decanted and the aqueous layer extracted with methylene chloride (3×15 ml). The combined organic phases were washed with water (30 ml) and brine (30 ml), dried over anhydrous sodium sulphate and evaporated at reduced pressure to give the free base form of vinblastine that was collected after precipitation with a mixture of ethyl ether and hexane (1:1). Yield 1.31 g (1.61 mmol, 98% yield).

Vinblastine free base (60 mg, 0.06 mmol) was dissolved in dry methanol (5 ml), added to a solution of sodium methoxide (NaOCH₃, 190 mg; dry methanol 7 ml) and kept under stirring for 3 h at room temperature. After that the reaction mixture was diluted with methylene chloride and washed with cold brine, then twice with aqueous solution of NaHCO₃ and finally with brine again until pH 7-8. The organic layer was separated off, dried over anhydrous sodium sulphate and the solvent was removed at reduced pressure to give an off-white crystalline solid of 4-deacetylvinblastine (21) (46 mg). TLC on kieselgel plate (Merck), eluting system methylene chloride/methanol (90/10 v/v) Rf=0.34.

Compound (21) (0.2 g, 0.26 mmol) in dry pyridine (4.5 ml) was treated with a solution of Fmoc-Leu-Cl (0.385 g, 1.04 mmol) dissolved in anhydrous methylene chloride (1.9 ml). The reaction mixture was kept at room temperature for 12 h. After that water (0.5 ml) was added to the reaction mixture to destroy the excess acid chloride and the solvent was evaporated at reduced pressure. The residue was partitioned between ethyl acetate and saturated NaHCO₃ and washed with brine. The organic phase was dried over anhydrous sodium sulphate and then the solvent was removed at reduced pressure. The residue was dissolved in a mixture of piperidine (1.3 ml) and dry DMF (2.7 ml) at room temperature. After 30 minutes the solvent was removed at reduced pressure and the crude oil was purified by flash chromatography on silica gel using as eluting system chloroform and methanol (95/5 v/v) to afford the title compound (23) (0.178 g, 62% yield). TLC on kieselgel plate (Merck), eluting system methylene chloride/methanol (90/10 v/v) Rf=0.3.

### EXAMPLE 7

### 4-Deacetyl-4-O-(6-aminohexanoyl-leucyl-glycyl-leucyl-leucyl)-vinblastine trifluoroacetate (1′c)

N-t-Butoxycarbonyl-6-aminohexanoyl-leucyl-glycyl-leucyl (0.17 g, 0.32 mmol) was dissolved in dry dimethylformamide (6 ml) together with 4-deacetyl-4-O-Leucyl-vinblastine (23) (0.28 g, 0.32 mmol), TBTU (0.1 g, 0.32 mmol) and HOBt (0.05 g, 0.32 mmol). After addition of diisopropylethylamine (0.1 ml, 0.26 mmol) the reaction mixture was stirred for 48 h. The solvent was then removed at reduced pressure and the residue partitioned between methylene chloride and saturated NaHCO₃. The organic layer was washed again with saturated NaHCO₃ and finally with brine. After drying over anhydrous sodium sulphate the solvent was removed at reduced pressure to give a crude oil which was purified by flash chromatography using as eluting system a mixture of chloroform/methanol (95/5 v/v) to afford 4-deacetyl-4-O-(t-butoxycarbonyl-6-aminohexanoyl-leucyl-glycyl-leucyl)-vinblastine (24) (0.32 g, 75% yield). TLC on kieselgel plate (Merck), eluting system methylene chloride/methanol (90/10 v/v) Rf=b 0.59.

Compound (24) was treated with trifluoroacetic (2.5 ml) acid 50% in methylene chloride. After 2 h stirring at room temperature the solvent was removed at reduced pressure and the residue was treated twice with toluene and taken to dryness to give the title compound (1c′) as di-trifluoroacetate salt.

### EXAMPLE 8

### Copolymer of N-(2-hydroxypropyl)methacryloylamide, 4-O-[N-methacryloyl-glycyl-6-aminohexanoy-leucyl-glycyl-leucyl-leucyl)-vinblastine-and N-(2-hydroxy-propyl) methacryloylglycinamide (A1c)

Compound (1′c: 0.3 g, 0.22 mmol) prepared as described in Example 7 was dissolved in dry dimethylsulfoxide (1.5 ml), added to a solution of (P1′) (1.5 g) dissolved in dry dimethylsulfoxide (6 ml), followed by diisopropylethylamine (0.7 ml, 0.25 mmol). After 24 h stirring at room temperature 1-amino-2-propanol (0.1 ml) was added to the reaction mixture and, after 1 h, the reaction mixtures was poured into ethyl acetate (100 ml). The precipitate containing (A1c) was filtered and washed with ethyl ether, then was dissolved in 15 ml of water (HPLC grade) and purified on a G-25 size exclusion cartridge (7 runs, eluent: water). Pooled fractions yielded, upon lyophilization, the title compound (A1c) (1.2 g, 78% yield).

### Antitumor Activity

Campound (A1a) was tested on human colon carcinoma (HT29) transplanted in nude mice, in comparison with the free drug 7-ethyl-10-hydroxycamptothecin (12) by i.v. route. A1a was found non toxic at all tested doses and gave 98% tumor inhibition at the highest tested dose of 20 mg/kg (Table 1).

TABLE 1
Antitumor Activity of Ala on human colon carcinoma
(HT29) in comparison with 7-ethyl-10-hydroxycamptothecin
(12). Treatment i.v. q4dx6.

| | Dose | Total Dose | TI | |
|---|---|---|---|---|
| Compound | mg/kg | mg/kg | % | Tox |
| Ala | 10 | 60 | 85 | 0/7 |
| | 20 | 120 | 97 | 0/7 |
| | 30 | 180 | 97 | 0/7 |
| | 40 | 240 | 97 | 0/7 |
| 12 | 10 | 60 | 67 | 0/7 |
| | 20 | 120 | 97 | 3/7 |

Tumor fragment were implanted sc.
Treatment started when tumor was palpable.
TI% (tumor inhibition %) was calculated at day 46.
Tox number of mice died for toxicity/total number mice

1. A polymeric drug-conjugate of formula (A)
P-[W₂]ₚ-S₀-[W₁]ᵣ[D] (A)
wherein:
P is a water soluble polymer;
[W₁] is a residue of formula —HN-Z-CO— in which Z₁ represents a linear or branched C₂-C₁₂ alkylene chain or the residue of formula —C₆H₄—CH₂—O—;
[W₂] is a residue of formula —HN-Z₂-CO— in which Z₂ represents a C₂-C₁₂ linear or branched alkylene chain;
p and r are 0 or 1;
S₀ is a peptide residue selectively cleavable at the tumor site by the action of matrix metalloproteinases and [D] is the residue of an antitumor agent:

2. A polymeric conjugate according to claim 1 wherein [D] is the residue of an antitumor agents bearing functional groups for the attachment of the linker W₁ or the peptide S₀ portion of the conjugate of formula (A) as defined in claim 1; r is 0 or [W₁] is the self-imolative p-aminobenzyloxycarbonyl linker, [W₂] is the residue of 6-aminohexanoic acid; and [P] is poly-glutamic acid, a carboxylated dextrane, carboxylated polyethylenglycols or a polymer based on N-(2-hydroxypropyl)methacryloylamide.

3. A polymeric conjugate according to claim 1 or 2 in which the peptide S₀ comprises sequences from four to five natural or synthetic amino acids.

4. A polymeric conjugate according to claim 3 wherein S₀ represents a sequence of formula:

| | |
|---|---|
| Met(O)-Gly-Cys(Bn)-Leu, | (SEQ ID NO: 1) |
| Met(O)-Gly-Cys(Bn)-Gly, | (SEQ ID NO: 2) |
| Met(O)-Gly-Cys(IBn)-Gly-Leu, | (SEQ ID NO: 3) |
| Met(O)-Gly-Cys(Bn)-Trp-Gly, | (SEQ ID NO: 4) |
| Met(O)-Gly-Cys(Bn)-pFIF-Gly, | (SEQ ID NO: 5) |
| Met(O)-Gly-Cys(Bfl)-Gly-Gly, | (SEQ ID NO: 6) |
| Met(O)-Gly-Cys(Bn)-Leu-Gly, | (SEQ ID NO: 7) |
| Smc-Gly-Cys(Bn)-Leu, | (SEQ ID NO: 8) |
| Smc-Gly-Cys(Bn)-Trp, | (SEQ ID NO: 9) |
| Smc-Gly-Cys(Bn)-pFF, | (SEQ ID NO: 10) |
| Smc-Gly-Cys(Bn)-Gly, | (SEQ ID NO: 11) |
| Smc-Gly-Cys(Bn)-Trp-Gly, | (SEQ ID NO: 12) |
| Smc-Gly-Cys(Bn)-pFF-Gly, | (SEQ ID NO: 13) |
| Smc-Gly-Cys(Bn)-Gly-Gly, | (SEQ ID NO: 14) |
| Smc-Gly-Cys(Bn)-Leu-Gly, | (SEQ ID NO: 15) |
| Smc-Gly-Leu-Trp, | (SEQ ID NO: 16) |
| Smc-Gly-Tha-Trp, | (SEQ ID NO: 17) |
| Smc-Gly-Met-Trp, | (SEQ ID NO: 18) |
| Smc-Gly-Tha-Trp-Gly, | (SEQ ID NO: 19) |
| Smc-Gly-Met-Trp-Gly, | (SEQ ID NO: 20) |
| Leu-Gly-Cys(lRn)-Leu, | (SEQ ID NO: 21) |
| Leu-Gly-Cys(Bn)-Gly, | (SEQ ID NO: 22) |
| Leu-Gly-Cys(Bn)-Leu-Gly, | (SEQ ID NO: 23) |
| Leu-Gly-Cys(Bn)-Gly-Gly, | (SEQ ID NO: 24) |
| Leu-Gly-Leu-Leu, | (SEQ ID NO: 25) |
| Leu-Gly-Leu-Trp, | (SEQ ID NO: 26) |
| Leu-Gly-Leu-Leu-Gly or | (SEQ ID NO: 27) |
| Leu-Gly-Leu-Trp-Gly. | (SEQ ID NO: 28) |

5. A polymeric conjugate according to claim 4 wherein S₀ represents a sequence of formula: Met(O)-Gly-Cys(Bn)-Leu (SEQ ID NO: 1), Met(O)-Gly-Cys(Bn)-Gly (SEQ ID NO: 2), Met(O)-Gly-Cys(Bn)-Gly-Gly (SEQ ID NO: 6), Met(O)-Gly-Cys(Bn)-Leu-Gly (SEQ ID NO: 7), Smc-Gly-Cys(Bn)-Leu (SEQ ID NO: 8), Smc-Gly-Cys(Bn)-Gly (SEQ ID NO: 11), Smc-Gly-Cys(Bn)-Gly-Gly (SEQ ID NO: 14), Smc-Gly-Cys(Bn)-Leu-Gly (SEQ ID NO: 15), Leu-Gly-Cys(Bn)-Leu (SEQ ID NO: 21), Leu-Gly-Cys(Bn)-Gly (SEQ ID NO: 22), Leu-Gly-Cys(Bn)-Leu-Gly (SEQ ID NO: 23), Leu-Gly-Cys(Bn)-Gly-Gly (SEQ ID NO: 24), Leu-Gly-Leu-Leu (SEQ ID NO: 25) or Leu-Gly-Leu-Leu-Gly (SEQ ID NO: 27).

6. A polymeric conjugate according to any one of the preceding claims wherein the antitumor agent [D] is a cytotoxic agent belonging to the class of vinca alkaloids, anthracyclines, taxanes, cytotoxic nucleosides, camptothecins, podophyllotoxins, or alkylating agents.

7. A polymeric conjugate according to claim 6 wherein the antitumor agent [D] is 4-deacetyl-vinblastine, 4-deacetyl-vincristine, vindesine, doxorubicin, 4′-epidoxorubicin, daunorubicin, 4-demethoxy-daunorubicin, 4′-deoxy-4′-iododoxorubicin, 3′-(2-methoxymorpholino) doxorubicin, paclitaxel, docetaxel, 5-fluorouracil, camptothecin, 7-ethyl-10-hydroxycamptothecin, 9-aminocamptothecin, etoposide or estramustine.

8. A polymeric conjugate according to any one of the preceding claims wherein [P] is a water soluble polymer based on N-(2-hydroxypropyl)methacryloylamide.

9. A process for preparing a polymeric conjugate as defined in claim 1, which process comprises reacting a compound of general formula (1) or a corresponding salt derivative of formula (1′):
H-[W₂]ₚ-S₀-[W₁]ᵣ-[D] (1)
RH H-[W₂]ₚ-S₀-[W₁]ᵣ-[D] (1′)
wherein [W₁], [W₂], p, r, S₀ and [D] are as defined in claim 1; and RH is an acid, with a polymer P1 bearing suitable functional groups for the coupling with compounds (1) or (1′).

10. A process according to claim 9 in which the suitable functional groups on polymer [P1] for the attachment of a compound (1) or (1′) as defined in claim 9 comprise carboxyl groups or activated carboxyl groups.

11. An antitumor derivative of formula (1) or the corresponding salt derivative of formula (1′) as defined in claim 9.

12. A process for preparing a compound of formula (1) or (1′) as defined in claim 11, which process comprises
removing the N-protecting group from a derivative of formula (2);
R₂-[W₂]ₚ-S₀-[W₁]ᵣ-[D] (2)
wherein [W₁], [W₂], p, r, [D] and S₀ are as defined in claim 9, and R₂ represents an amino-protecting group and
optionally converting a resultant compound of general formula (1′) into the corresponding free amino derivative (1) by mild basic treatment.

13. A process according to claim 12 in which the removal of the N-protecting group is carried out in acidic conditions, and R₂ represents Boc (tert-butoxycarbonyl), Fmoc, triphenylsilyl, diphenylmethylene or triphenylmethyl group.

14. A polymeric drug-conjugate according to claim 1 which comprises a water soluble polymer [P] consisting of:
(i) from 85 to 97 mol % of N-(2-hydroxypropyl)methacryloylamide units represented by formula (25) 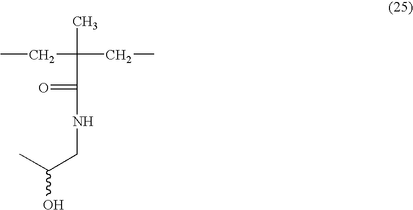
(ii) from 3 to 15 mol % of units represented by formula (26) 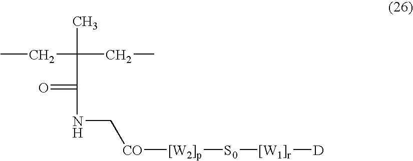
in which [W₁], [W₂], p, r, [D] and S₀ are as defined in claim 1,
(iv) from 0 to 12 mol % of N-methacryloyl-glycine or N-(2-hydroxypropyl) methacryloyl-glycinamide units represented by formula (27) 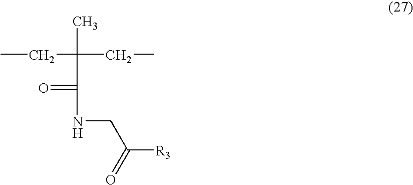
wherein R₃ represents a hydroxy group or a residue of formula
—NH—CH₂—CH(OH)—CH₃.

15. A process for preparing a drug-conjugate as defined in claim 14, which process comprises reacting a compound of formula (1) or (1′) as defined in claim 9 with an activated water soluble polymer (P1′) consisting essentially of:
(i) from 85 to 97 mol % of N-(2-hydroxypropyl)methacryloylamide units represented by formula (25) as defined in claim 14, and
(ii) from 3 to 15 mol % of N-methacryloyl-glycyl units represented by formula (28) 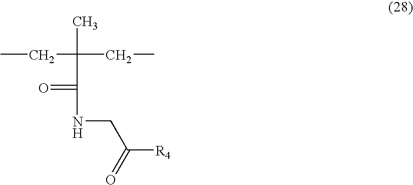
wherein R₄ is the residue of an active ester, and optionally displacing the remaining active ester groups with 1-amino-2-propanol.

16. A pharmaceutical composition comprising a pharmaceutically acceptable diluent or carrier and, as active ingredient, a polymeric conjugate as defined in any one of claims 1 to 8 or 14 or a compound of formula (1) or (1′) as defined in claim 11.

17. A polymeric conjugate as defined in any one of claims 1 to 18 or 14 or a compound of formula (1) or (1′) as defined in claim 11 for use in a method of treatment of the human or animal body by therapy.

18. Use of a polymeric conjugate as defined in any one of claims 1 to 8 or 14 or a compound of formula (1) or (1′) as defined in claim 11 13 in the manufacture of a medicament for treating leukemia or a solid tumor.

19. Use according to claim 18, wherein the solid tumor is a colon, colo-rectal, ovarian, mammary, prostate, lung or kidney tumor or a melanoma.
